(19) United States
(12) Patent Application Publication
(10) Pub. No.: US 2006/0211699 A1
(43) Pub. Date: Sep. 21, 2006

(54) Quinoxaline dihydrohalide dihydrates and synthetic methods therefor

(75) Inventors: Silvio Iera, Montreal (CA); Abdolsamad Tadayon, Kirkland (CA); John Hamilton Sellstedt, Iden Prairie, MN (US); Subodh Deshmukh, White Plains, NY (US)

Correspondence Address: WOODCOCK WASHBURN LLP, ONE LIBERTY PLACE - 46TH FLOOR, PHILADELPHIA, PA, 19103, US

(73) Assignee: Wyeth, Madison, NJ (US)

(21) Appl. No.: 11/371,150

(22) Filed: Mar. 7, 2006

Related U.S. Application Data
(60) Provisional application No. 60/659,228, filed on Mar. 7, 2005.

(51) Int. Cl. A61K 31/496; C07D 403/02; A61K 31/498; C07D 403/14

(52) U.S. Cl. 514/249; 514/254.06; 544/368; 544/333

(57) ABSTRACT

Crystalline polymorph forms of Gonadotropin Releasing Hormone receptor antagonists, including crystalline polymorphs of quinoxaline dihydrohalide dihydrates, in particular crystalline polymorphs of 6-({4-[2-(4-tert-butylphenyl)-1H-benzimidazol-4-yl]-piperazin-1-yl}methyl)-quinoxaline dihydrochloride dihydrate, methods of making the same, as well as pharmaceutical compositions, and dosage forms containing them are disclosed.

### CROSS REFERENCE TO RELATED APPLICATIONS

The present application claims benefit of U.S. Provisional Application Ser. No. 60/659,228 filed Mar. 7, 2005, the disclosure of which is hereby incorporated by reference in its entirety.

### FIELD OF THE INVENTION

The present invention relates to crystalline polymorphs of Gonadotropin Releasing Hormone (“GnRH”) receptor antagonists, including crystalline polymorphs of quinoxaline dihydrohalide dihydrates, in particular to crystalline polymorphs of 6-({4-[2-(4-tert-butylphenyl)-1H-benzimidazol-4-yl]-piperazin-1-yl}methyl)-quinoxaline dihydrochloride dihydrate, methods of making the same, as well as pharmaceutical compositions, and dosage forms containing them.

### BACKGROUND OF THE INVENTION

GnRH is a decameric peptide released from the hypothalamus. In the anterior pituitary gland, GNRH activates the GnRH receptor. Activation of the GnRH receptor triggers the release of follicle stimulating hormone (FSH) and leuteinizing hormone (LH). FSH and LH stimulate the biosynthesis and release of sex steroids in the gonads of both genders.

Typically, this is desirable, but certain sex hormone dependent pathological conditions exist where it would be beneficial to prevent activation of the GnRH receptor. For example, inhibition of the GnRH receptor can lead to a large drop in sex steroid production, which in turn can alleviate sex hormone dependent pathological conditions such as prostate cancer, endometriosis, uterine fibroids, uterine cancer, breast cancer, ovarian cancer, testicular cancer, or primary hirsutism. Moreover, there are other situations where it would be beneficial to prevent activation of the GnRH receptor, such as during some points of the in vitro fertilization process, such as to, for example, prevent LH surge.

Most currently marketed GnRH therapeutics are peptides as such, they are not orally bioavailable and must be administered via parenteral means such as intravenous, subcutaneous or intramuscular injection. Thus, non-peptide GnRH antagonists would be of significant benefit.

Concurrently filed U.S. Application Ser. No. 60/580,640 and U.S. Application Ser. No.60/580,665, the disclosures of which are hereby incorporated by reference in their entireties, teach, inter alia, GnRH receptor antagonists and methods of making GnRH receptor antagonists. Crystalline forms of GnRH receptor antagonists and procedures for synthesizing the same would be desirable. U.S. Application Ser. No. 60/580,640 is available as the priority document of WO/2006/009734. U.S. Application Ser. No.60/580,665 is available as the priority document of WO/2006/009736. The disclosures of WO/2006/009734 and WO/2006/009736 are also hereby incorporated by reference in their entireties.

### SUMMARY OF THE INVENTION

The present invention provides crystalline polymorphs of GnRH receptor antagonists, and in particular to crystalline polymorphs of quinoxaline dihydrohalide dihydrates. In one embodiment, the invention is directed to crystalline polymorphs of 6-({4-[2-(4-tert-butylphenyl)-1H-benzimidazol-4-yl]-piperazin-1-yl}methyl)-quinoxaline dihydrohalide dihydrate. In another embodiment, the present invention is directed to crystalline polymorphs of 6-({4-[2-(4-tert-butylphenyl)-1H-benzimidazol-4-yl]-piperazin-1-yl}methyl)-quinoxalin dihydrochloride dihydrate.

The present invention also provides methods of preparing crystalline polymorphs of GnRH receptor antagonists, including methods of preparing crystalline polymorphs of quinoxaline dihydrohalide dihydrates. In one embodiment, the present invention also provides methods of preparing crystalline polymorphs of 6-({4-[2-(4-tert-butylphenyl)-1H-benzimidazol-4-yl]-piperazin-1-yl}methyl)-quinoxaline dihydrohalide dihydrate, in particular, methods of preparing crystalline polymorphs of 6-({4-[2-(4-tert-butylphenyl)-1H-benzimidazol-4-yl]-piperazin-1-yl}methyl)-quinoxaline dihydrochloride dihydrate, and in particular, Forms A, B and C. The present invention also provides pharmaceutical compositions comprising the compounds of the invention.

In other embodiments, the present invention provides methods of treating patients suspected of suffering from sex hormone dependent pathological conditions such as prostate cancer, endometriosis, uterine fibroids, uterine cancer, breast cancer, ovarian cancer, testicular cancer, primary hirsutism, or luteinizing hormone surge, comprising administering to a patient an effective amount of compounds of the invention.

### BRIEF DESCRIPTION OF THE DRAWINGS

FIGS. 1a and 1b are thermogravimetric analyses (TGA) of seeds (FIG. 1a) and crystals (FIG. 1b) of 6-({4-[2-(4-tert-butylphenyl)-1H-benzimidazol-4-yl]-piperazin-1-yl}methyl)-quinoxaline dihydrochloride dihydrate. The crystals are of form A. While the samples were heated from 35° C. to 300° C. at a scan rate of 20° C./min., approximately 7.6% of solvent content (water) was lost. Crystals were generated by seeding. The scans show that the resulting crystals and the seeds have the same thermogravimetric behavior.

FIGS. 2a and 2b show X-Ray diffraction (XRD) patterns of samples of 6-({4-[2-(4-tert-butylphenyl)-1H-benzimidazol-4-yl]-piperazin-1-yl}methyl)-quinoxaline dihydrochloride dihydrate. Both samples are form A. The sample illustrated in FIG. 2b is 6-({4-[2-(4-tert-butylphenyl)-1H-benzimidazol-4-yl]-piperazin-1-yl}methyl)-quinoxaline dihydrochloride dihydrate crystal Form A and was generated by seeding a solution with the sample illustrated in FIG. 2a. The sample in FIG. 2a is seeds of 6-({4-[2-(4-tert-butylphenyl)-1H-benzimidazol-4-yl]-piperazin-1-yl}methyl)-quinoxaline dihydrochloride dehydrate. The scans are showing that the resulting crystals and the seeds have the same XRD patterns. The relative intensities of the XRD peaks can very depending on the sample preparation technique and crystal size distribution, the sample mounting procedure, and the particular instrument employed. Moreover, some peaks may appear or disappear depending on the type of machine or the settings (for example whether a Ni filter is used or not). In the present invention, the patterns were obtained from a Bruker D8 advance machine with no Ni filter.

FIG. 3 shows different XRD patterns of 6-({4-[2-(4-tert-butylphenyl)-1H-benzimidazol-4-yl]-piperazin-1-yl}methyl)-quinoxaline dihydrochloride dehydrate. The sample in the lower scan is Form A, the sample in the middle scan is Form C, and the sample in the top scan is Form B.

### DETAILED DESCRIPTION OF ILLUSTRATIVE EMBODIMENTS

In accordance with the present invention, an “alcohol” is a polar solvent that at least partially dissolves the starting material and product. Representative alcohols include C₁-C₆ alcohols, with ethanol preferred.

The term “acid”, as used herein, refers to a compound that is capable of dissociating in water and is a proton donor. Preferably, the acid is hydrochloric acid.

The term “halo”, as used herein, includes chlorine, fluorine, bromine, and iodine.

In one aspect, the present invention relates to crystalline polymorphs of GnRH receptor antagonists of formula I: 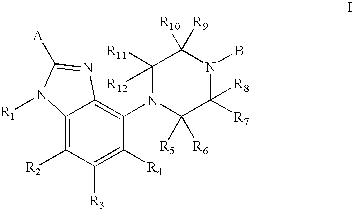
wherein:
A is aryl or heteroaryl;
B is (CR₁₃R₁₄)ₖ-D;
D is H, alkyl, cycloalkyl, heterocycloalkyl, aryl, or heteroaryl;
k is 0, 1, 2, or 3;
R₁ is H, the tautomeric form, or alkyl;
R₂, R₃, and R₄ are, independently, H, alkyl, halogen, or OR₁;
R₅, R₆, R₇, R₈, R₉, R₁₀, R₁₁, and R₁₂, are, independently, H, alkyl, alkenyl, or alkynyl;
R₁₃ and R₁₄ are, independently at each occurrence, H or alkyl.

In another aspect, the present invention provides crystalline polymorphs of the dihydrohalide dihydrate forms of compounds of formula I. In one embodiment, crystalline polymorphs of the dihydrohalide dihydrate forms of compounds of formula I include crystalline polymorphs of 6-({4-[2-(4-tert-butylphenyl)-1H-benzimidazol-4-yl]-piperazin-1-yl}methyl)-quinoxaline dihydrohalide dihydrate, and in particular crystalline polymorphs of 6-({4-[2-(4-tert-butylphenyl)-1H-benzimidazol-4-yl]-piperazin-1-yl}methyl)-quinoxaline dihydrochloride dihydrate, including crystalline polymorph forms A, B, and C. 6-({4-[2-(4-tert-butylphenyl)-1H-benzimidazol-4-yl]-piperazin-1-yl}methyl)-quinoxaline dihydrochloride dihydrate has a formula II:

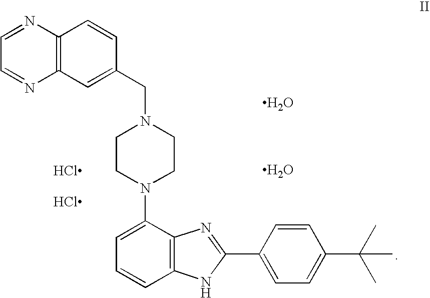

In another aspect, the present invention relates to methods of making crystalline polymorphs of formula I, and methods of making crystalline polymorphs of dihydrohalide dihydrate forms of compounds of formula I. In another aspect, the present invention is directed to methods of making crystalline polymorphs of 6-({4-[2-(4-tert-butylphenyl)-1H-benzimidazol-4-yl]-piperazin-1-yl}methyl)-quinoxaline dihydrohalide dihydrate. In another aspect, the present invention provides methods of making crystalline polymorphs of 6-({4-[2-(4-tert-butylphenyl)-1H-benzimidazol-4-yl]-piperazin-1-yl}methyl)-quinoxaline dihydrochloride dihydrate, and in particular, Forms A, B and C.

Compounds of formula I can be prepared, for example, by generally following the procedures described in U.S. Application Ser. No. 60/580,640 and U.S. Application Ser. No. 60/580,665. Dihydrohalide dihydrate crystalline forms of compounds of formula I can be prepared, for example, as generally shown in Scheme 1, where X is a halogen, preferably Cl.

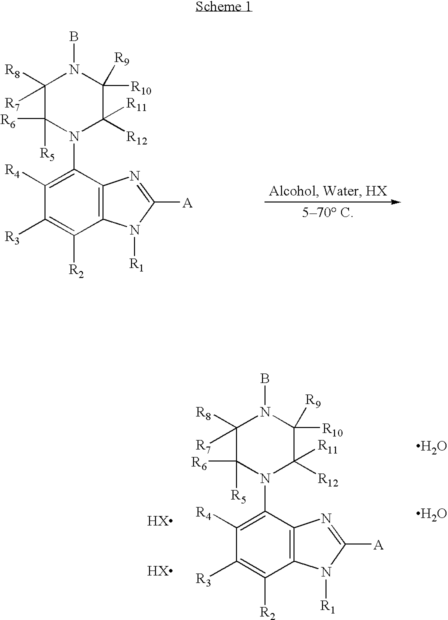

Crystalline polymorphs of 6-({4-[2-(4-tert-butylphenyl)-1H-benzimidazol-4-yl]-piperazin-1-yl}methyl)-quinoxaline dihydrohalide dihydrates, where A is 4-tert-butylphenyl, B is quinoxalin-6-ylmethyl, and R₁, R₂, R₃, R₄, R₅, R₆, R₇, R₈, R₉, R₁₀, R₁₁, and R₁₂ are each H, can be prepared, for example, as shown in Scheme 2, where X is a halogen, preferably Cl. Crystalline polymorphs of 6-({4-[2-(4-tert-butylphenyl)-1H-benzimidazol-4-yl]-piperazin-1-yl}methyl)-quinoxaline dihydrochloride dihydrate can be prepared, for example as shown in Scheme 2, and described below, where X is Cl.

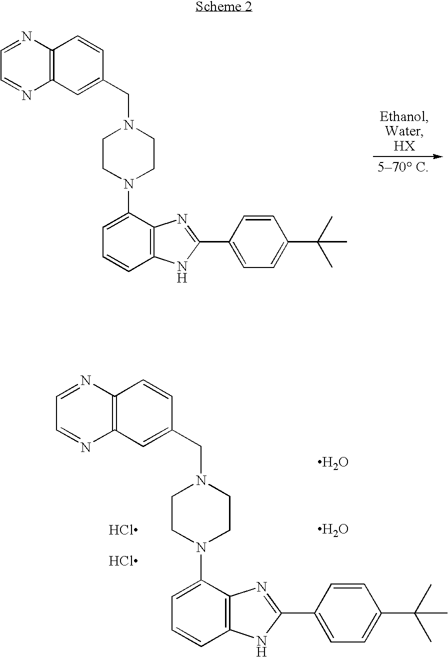

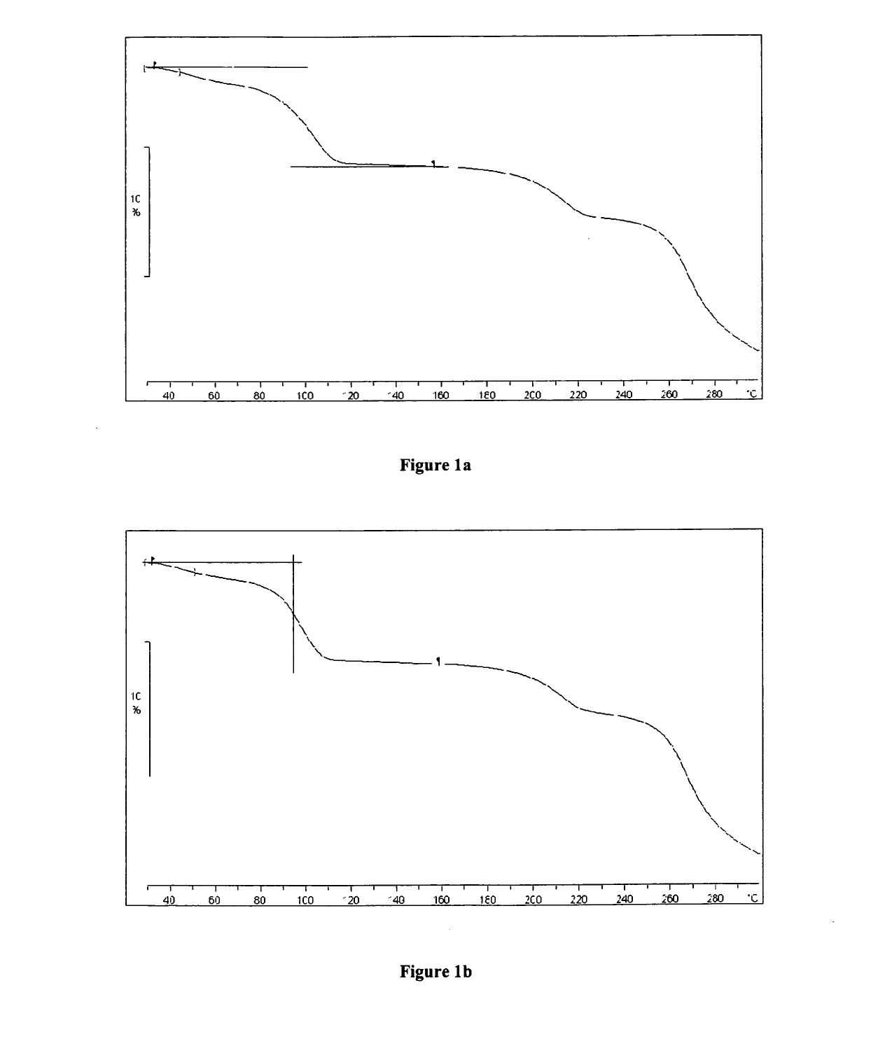

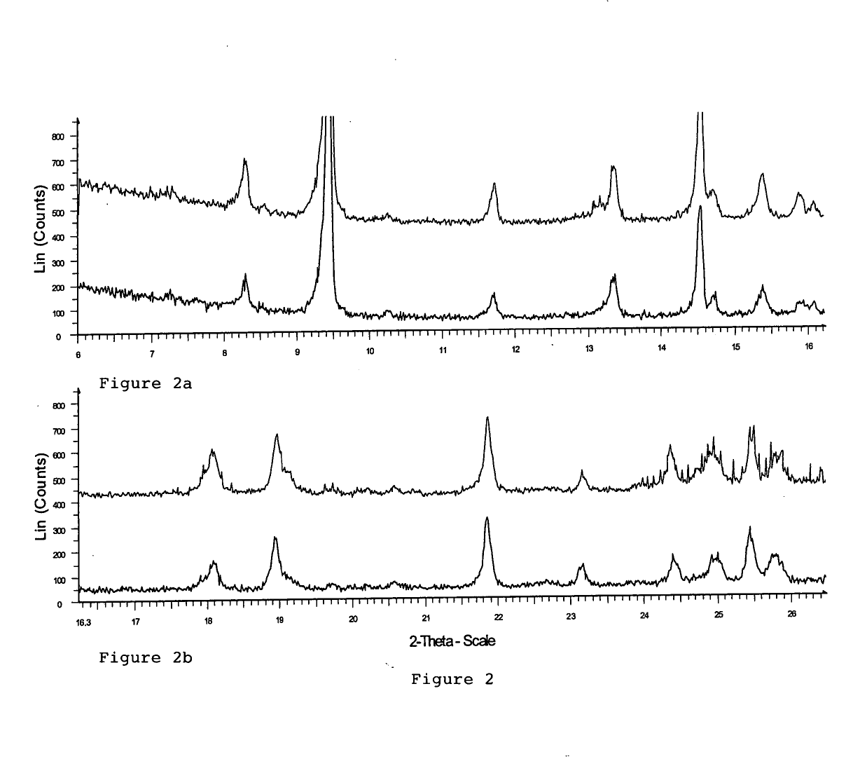

The free base is used as a starting material, to which is added ethanol in, for example, an oil bath. Water is then added and the suspension is stirred at about 67° C. until all solids dissolved. Aqueous HCl is added to the free base solution, with stirring. The bath temperature is then reduced to about 63° C. and seeds of Form A are added. The suspension is stirred for 30 min, wherein crystals are formed. The suspension is then cooled to room temperature for about 1.5 hr and then stirred for an additional 1.5 hr. The suspension is filtered (fast filtration) and dried in an oven at about 56° C. and under about 75 mm of water vacuum (gauage pressure) overnight. This yields 6-({4-[2-(4-tert-butylphenyl)-1H-benzimidazol-4-yl]-piperazin-1-yl}methyl)-quinoxaline dihydrochloride dihydrate Form A. FIG. 1 shows thermogravimetric scans of 6-({-4-[2-(4-tert-butylphenyl)-1H-benzimidazol-4-yl]-piperazin-1-yl}methyl)-quinoxaline dihydrochloride dihydrate Form A and seeds of 6-({-4-[2-(4-tert-butylphenyl)-1H-benzimidazol-4-yl]-piperazin-1-yl}methyl)-quinoxaline dihydrochloride dihydrate. FIG. 2 shows X-ray diffraction (XRD) scans comparing of 6-({-4-[2-(4-tert-butylphenyl)-1H-benzimidazol-4-yl]-piperazin-1-yl}methyl)-quinoxaline dihydrochloride dihydrate Form A to crystal seeds of compound.

Form A has an XRD pattern having peaks expressed in degrees 2θ as shown in Table 1.

TABLE 1
XRD patterns of Form A

| Angle 2-θ° | Intensity % |
|---|---|
| 7.232 | 13.9 |
| 8.275 | 22.9 |
| 9.442 | 100.0 |
| 10.225 | 6.8 |
| 11.714 | 15.2 |
| 13.150 | 8.7 |
| 13.357 | 19.5 |
| 14.539 | 41.1 |
| 15.394 | 17.1 |
| 15.895 | 11.8 |
| 16.101 | 9.2 |
| 18.076 | 16.8 |
| 18.959 | 20.8 |
| 19.680 | 5.0 |
| 20.165 | 4.9 |
| 20.578 | 5.9 |
| 21.876 | 24.8 |
| 23.170 | 9.4 |
| 24.033 | 5.4 |
| 24.389 | 14.4 |
| 24.948 | 18.3 |
| 25.829 | 13.9 |
| 26.930 | 12.6 |
| 29.309 | 11.3 |
| 30.482 | 8.9 |

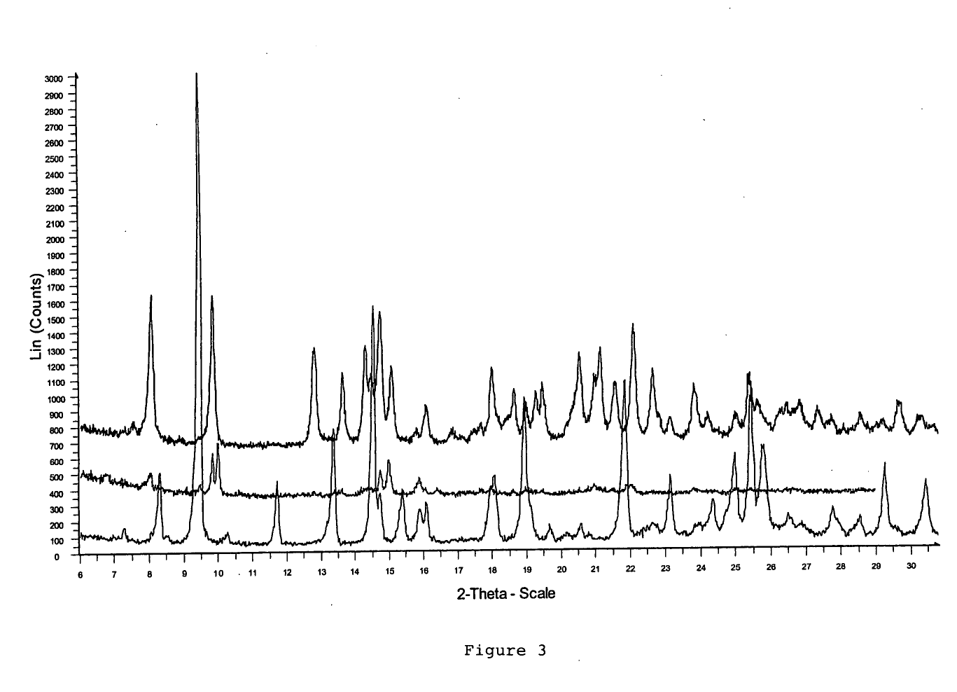

Other crystal forms of 6-({4-[2-(4-tert-butylphenyl)-1H-benzimidazol-4-yl]-piperazin-1-yl}methyl)-quinoxaline dihydrochloride dihydrate can be prepared by varying the reaction conditions described above. By following the above procedure but not seeding the solution, 6-({4-[2-(4-tert-butylphenyl)-1H-benzimidazol-4-yl]-piperazin-1-yl}methyl)-quinoxaline dihydrochloride dihydrate Form B is obtained. Form B presents a different XRD pattern from that of form A, which can be seen from FIG. 3. Alternatively, by following the above procedure but using more ethanol and less water, 6-({4-[2-(4-tert-butylphenyl)-1H-benzimidazol-4-yl]-piperazin-1-yl}methyl)-quinoxaline dihydrochloride dihydrate Form C, is obtained. Form C presents a different XRD pattern from that of Forms A and B, which can also be seen in FIG. 3. Form B and Form C have XRD pattern having peaks expressed in degrees 2θ as shown in Table 2 and Table 3, respectively.

TABLE 2
XRD patterns of Form B

| Angle 2-θ° | Intensity % |
|---|---|
| 7.514 | 22.4 |
| 8.031 | 100 |
| 9.821 | 99.9 |
| 12.782 | 66.8 |
| 13.619 | 51.3 |
| 14.282 | 68.1 |
| 14.491 | 50.3 |
| 14.708 | 88.4 |
| 15.05 | 55.4 |
| 16.073 | 30.8 |
| 16.833 | 15.4 |
| 17.99 | 53.7 |
| 18.624 | 40 |
| 18.979 | 31.8 |
| 19.269 | 39.2 |
| 19.453 | 44.3 |
| 20.532 | 62.4 |
| 21.122 | 63.6 |
| 21.566 | 43.8 |
| 22.086 | 79.9 |
| 22.652 | 52.1 |
| 23.152 | 21.8 |
| 23.845 | 39 |
| 24.22 | 25 |
| 25.026 | 24.1 |
| 25.417 | 49.2 |
| 25.649 | 31.4 |
| 26.281 | 25.9 |
| 26.837 | 31.3 |
| 27.367 | 25 |
| 27.744 | 22.7 |
| 28.588 | 23.3 |
| 29.229 | 19.7 |
| 29.697 | 30 |
| 30.307 | 20.8 |
| 30.656 | 14.9 |
| 31.128 | 17.8 |

TABLE 3
XRD patterns of Form C

| Angle 2-θ° | Intensity % |
|---|---|
| 6.737 | 51.8 |
| 7.981 | 47.9 |
| 9.440 | 33.1 |
| 9.809 | 82.8 |
| 9.980 | 100.0 |
| 13.579 | 21.0 |
| 14.713 | 53.1 |
| 14.976 | 69.2 |
| 15.857 | 40.0 |
| 16.399 | 23.6 |
| 17.952 | 22.1 |
| 18.606 | 18.5 |
| 18.985 | 18.5 |
| 20.950 | 27.2 |
| 21.983 | 25.4 |
| 25.029 | 18.2 |

This invention also provides methods of treating diseases and conditions in a mammal associated with activity of the GnRH receptor including, for example, prostate cancer, endometriosis, uterine fibroids, uterine cancer, breast cancer, ovarian caner, testicular caner, primary hirsutism, and lutenizing hormone (“LH”) surge. The term “treating”, as used herein, is intended to include preventing, inhibiting or otherwise alleviating a disease or condition of interest. The methods of the invention are preferably practiced with respect to a human, and generally comprise administering an effective amount of a compound of the invention to a mammal in need thereof.

The term “patient”, as used herein, refers to a mammal, preferably a human.

The terms “administer”, “administering”, or “administration”, as used herein, refer to either directly administering a compound or composition to a patient, or administering a prodrug derivative or analog of the compound to the patient, which will form an equivalent amount of the active compound or substance within the patient's body.

The term “carrier”, as used herein, shall encompass carriers, excipients, and diluents.

The term “effective amount” refers to an amount of a compound as described herein that is able to produce a stated result. For example, the term “effective amount” when used with respect to a particular disease or disorder can refer to an amount that is effective to at least partially inhibit, prevent, treat, or modulate the symptoms of that disease or disorder. This can include, for example, contacting cells, tissues, or receptors with compounds of the present invention.

The dosage amounts useful to treat, prevent, inhibit or alleviate each of the aforementioned conditions will vary with the severity of the condition to be treated and the route of administration. The dose and dose frequency will also vary according to age, body weight, response and past medical history of the individual human patient. In generally the recommended daily dose range for the conditions described herein lie within the range of 10 mg to about 1000 mg/day and more preferably within the range of about 15 mg to about 350 mg/day and still more preferably from about 15 mg to about 140 mg/day. In other embodiments of the invention the dosage will range from about 30 mg to about 90 mg/day. Dosage is described in terms of the free base and is adjusted accordingly for the dihydrochloride salt. In managing the patient, is generally preferred that the therapy be initiated at a lower dose and increased if necessary. Dosages for non-human patients can be adjusted accordingly by one skilled in the art.

The phrase “pharmaceutically acceptable” refers to additives or compositions that are physiologically tolerable and do not typically produce an allergic or similar untoward reaction, such as gastric upset, dizziness and the like, when administered to an animal, such as a mammal (e.g., a human). For oral liquid pharmaceutical compositions, pharmaceutical carriers and excipients can include, but are not limited to water, glycols, oils, alcohols, flavoring agents, preservatives, coloring agents, and the like. Oral solid pharmaceutical compositions may include, but are not limited to starches, sugars, microcrystalline cellulose, diluents, granulating agents, lubricants, binders and disintegrating agents.

Any suitable route of administration can be employed for providing the patient with an effective amount of a compound of the invention. For example, oral, mucosal (e.g. nasal, sublingual, buccal, rectal or vaginal), parental (e.g. intravenous or intramuscular), transdermal, and subcutaneous routes, neat or in combination with conventional pharmaceutical carriers, can be employed. Preferred routes of administration include oral, transdermal and mucosal.

Applicable solid carriers can include one or more substances which may also act as flavoring agents, lubricants, solubilizers, suspending agents, fillers, glidants, compression aids, binders or tablet-disintegrating agents or encapsulating materials. They are formulated in conventional manner, for example, in a manner similar to that used for known antihypertensive agents, diuretics and β-blocking agents. Oral formulations containing the active compounds of this invention may comprise any conventionally used oral forms, including tablets, capsules, buccal forms, troches, lozenges and oral liquids, suspensions or solutions. In powders, the carrier is a finely divided solid, which is an admixture with the finely divided active ingredient. In tablets, the active ingredient is mixed with a carrier having the necessary compression properties in suitable proportions and compacted in the shape and size desired. The powders and tablets preferably contain up to 99% of the active ingredient.

Capsules may contain mixtures of the active compound(s) with inert fillers and/or diluents such as the pharmaceutically acceptable starches (e.g. corn, potato or tapioca starch), sugars, artificial sweetening agents, powdered celluloses, such as crystalline and microcrystalline celluloses, flours, gelatins, gums, etc.

Useful tablet formulations may be made by conventional compression, wet granulation or dry granulation methods and utilize pharmaceutically acceptable diluents, binding agents, lubricants, disintegrants, surface modifying agents (including surfactants), suspending or stabilizing agents, including, but not limited to, magnesium stearate, stearic acid, sodium lauryl sulfate, talc, sugars, lactose, dextrin, starch, gelatin, cellulose, methyl cellulose, microcrystalline cellulose, sodium carboxymethyl cellulose, carboxymethylcellulose calcium, polyvinylpyrrolidine, alginic acid, acacia gum, xanthan gum, sodium citrate, complex silicates, calcium carbonate, glycine, sucrose, sorbitol, dicalcium phosphate, calcium sulfate, lactose, kaolin, mannitol, sodium chloride, low melting waxes and ion exchange resins. Preferred surface modifying agents include nonionic and anionic surface modifying agents. Representative examples of surface modifying agents include, but are not limited to, poloxamer 188, benzalkonium chloride, calcium stearate, cetostearl alcohol, cetomacrogol emulsifying wax, sorbitan esters, colliodol silicon dioxide, phosphates, sodium dodecylsulfate, magnesium aluminum silicate, and triethanolamine. Oral formulations herein may utilize standard delay or time release formulations to alter the absorption of the active compound(s). The oral formulation may also consist of administering the active ingredient in water or fruit juice, containing appropriate solubilizers or emulisifiers as needed.

Liquid carriers may be used in preparing solutions, suspensions, emulsions, syrups and elixirs. The active ingredient of this invention can be dissolved or suspended in a pharmaceutically acceptable liquid carrier such as water, an organic solvent, a mixture of both or pharmaceutically acceptable oils or fat. The liquid carrier can contain other suitable pharmaceutical additives such as solubilizers, emulsifiers, buffers, preservatives, sweeteners, flavoring agents, suspending agents, thickening agents, colors, viscosity regulators, stabilizers or osmo-regulators. Suitable examples of liquid carriers for oral and parenteral administration include water (particularly containing additives as above, e.g. cellulose derivatives, preferably sodium carboxymethyl cellulose solution), alcohols (including monohydric alcohols and polyhydric alcohols, e.g. glycols) and their derivatives, and oils (e.g. fractionated coconut oil and arachis oil). For parenteral administration the carrier can also be an oily ester such as ethyl oleate and isopropyl myristate. Sterile liquid carriers are used in sterile liquid form compositions for parenteral administration. The liquid carrier for pressurized compositions can be halogenated hydrocarbon or other pharmaceutically acceptable propellant.

Liquid pharmaceutical compositions, which are sterile solutions or suspensions, can be utilized by, for example, intramuscular, intraperitoneal or subcutaneous injection. Sterile solutions can also be administered intravenously. Compositions for oral administration may be in either liquid or solid form.

Preferably the pharmaceutical composition is in unit dosage form, e.g. as tablets, capsules, powders, solutions, suspensions, emulsions, granules, or suppositories. In such form, the composition is sub-divided in unit dose containing appropriate quantities of the active ingredient; the unit dosage forms can be packaged compositions, for example, packeted powders, vials, ampoules, prefilled syringes or sachets containing liquids. The unit dosage form can be, for example, a capsule or tablet itself, or it can be the appropriate number of any such compositions in package form. Such unit dosage form may contain from about 1 mg/kg to about 250 mg/kg, preferably from 10 to 25 mg, and may be given in a single dose or in two or more divided doses. Such doses may be administered in any manner useful in directing the active compounds herein to the recipient's bloodstream, including orally, via implants, parenterally (including intravenous, intraperitoneal and subcutaneous injections), rectally, vaginally, and transdermally. Such administrations may be carried out using the present compounds, or pharmaceutically acceptable salts thereof, in lotions, creams, foams, patches, suspensions, solutions, and suppositories (rectal and vaginal).

In some cases it may be desirable to administer the compounds directly to the airways in the form of an aerosol. For administration by intranasal or intrabrochial inhalation, the compounds of this invention may be formulated into an aqueous or partially aqueous solution.

The compounds of this invention may be administered parenterally or intraperitoneally. Solutions or suspensions of these active compounds as a free base or pharmaceutically acceptable salt may be prepared in water suitably mixed with a surfactant such as hydroxyl-propylcellulose. Dispersions may also be prepared in glycerol, liquid polyethylene glycols and mixtures thereof in oils. Under ordinary conditions of storage and use, these preparations contain a preservative to inhibit the growth of microorganisms.

The pharmaceutical forms suitable for injectable use include sterile aqueous solutions or dispersions and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), suitable mixtures thereof, and vegetable oils.

The compounds of this invention can be administered transdermally through the use of a transdermal patch. For the purposes of this disclosure, transdermal administrations are understood to include all administrations across the surface of the body and the inner linings of bodily passages including epithelial and mucosal tissues. Such administrations may be carried out using the present compounds, or pharmaceutically acceptable salts thereof, in lotions, creams, foams, patches, suspensions, solutions, and suppositories (rectal and vaginal).

Transdermal administration may be accomplished through the use of a transdermal patch containing the active compound and a carrier that is inert to the active compound, is non-toxic to the skin, and allows delivery of the agent for systemic absorption into the blood stream via the skin. The carrier may take any number of forms such as creams and ointments, pastes, gels and occlusive devices. The creams and ointments may be viscous liquid or semisolid emulsions of either the oil-in-water or water-in-oil type. Pastes comprised of absorptive powders dispersed in petroleum or hydrophilic petroleum containing the active ingredient may also be suitable. A variety of occlusive devices may be used to release the active ingredient into the blood stream, such as a semi-permeable membrane covering a reservoir containing the active ingredient with or without a carrier, or a matrix containing the active ingredient. Other occlusive devices are known in the literature.

The compounds of this invention may be administered rectally or vaginally in the form of a conventional suppository. Suppository formulations may be made from traditional materials, including cocoa butter, with or without the addition of waxes to alter the suppository's melting point, and glycerin. Water soluble suppository bases, such as polyethylene glycols of various molecular weights, may also be used.

The following examples are illustrative, but are not meant to be limiting of the present invention.

### EXAMPLES

### Example 1

### 6-({4-[2-(4-tert-butylphenyl)-1H-benzimidazol-4-yl]-piperazin-1-yl}methyl)-quinoxaline dihydrochloride dihydrate Form A

6-({(2S)-4-[2-(4-tert-butylphenyl)-1H-benzimidazol-4-yl]-2-methylpiperazin-1-yl}methyl)-quinoxaline free base (1 g) was added to 7.2 ml of ethanol (99.5% EtOH, toluene 0.5%) in a 20 ml vial in an oil bath; 1.6 ml of water was added to the vial. The suspension was stirred (magnetic) at 67° C. (bath temperature) until all solids dissolved (15 min). In a different vial, 431 mg aqueous HCl (37% solution) was added to 1 ml of ethanol. The acid solution was added to the free base solution in 10 min while the solution was stirred. Bath temperature was reduced to 63° C. and seeds of form A were added. The suspension was stirred for 30 min; crystals were formed. The suspension was then cooled to room temperature in 1.5 hr and then stirred for an additional 1.5 hr. The suspension was filtered (fast filtration) and dried in an oven at 56° C. and 75 mm of water vacuum overnight. 92.7% recovered, including HCl and water. Water content was 7.5% by TGA, 8.5% by Karl Fischer method.

### Example 2

### 6-({4-[2-(4-tert-butylphenyl)-1H-benzimidazol-4-yl]-piperazin-1-yl}methyl)-quinoxaline dihydrochloride dihydrate Form B

The title compound was made by following the procedure of example 1, except that the solution was not seeded.

### Example 3

### 6-({4-[2-(4-tert-butylphenyl)-1H-benzimidazol-4-yl]-piperazin-1-yl}methyl)-quinoxaline dihydrochloride dihydrate Form C

The title compound was made by following the procedure of example 1, except that the volume of ethanol was increased (14 volumes) and the volume of water decreased (0.1 volume).

The present invention also provides a dihydrohalide dihydrate salt of a compound of formula I (wherein A, B, R₁, R₂, R₃, R₄, R₅, R₆, R₇, R₈, R₉, R₁₀, R₁₁, and R₁₂, are as defined above), preferably in crystalline form. The present invention also provides a dihydrochloride dihydrate salt of a compound of formula I (wherein A, B, R₁, R₂, R₃, R₄, R₅, R₆, R₇, R₈, R₉, R₁₀, R₁₁, and R₁₂, are as defined above), preferably in crystalline form. The present invention also provides a method comprising reacting a compound of formula I (wherein A, B, R₁, R₂, R₃, R₄, R₅, R₆, R₇, R₈, R₉, R₁₀, R₁₁, and R₁₂, are as defined above) with an alcohol, water, and an acid (preferably a hydrohalic acid, advantageously hydrochloric acid). The method may also comprise seeding the reaction product with a dihydrohalide dihydrate salt of the compound of formula I. The present invention also provides a pharmaceutical composition comprising a compound of the invention and a pharmaceutically acceptable carrier or excipient. The invention also provides use of a compound of the invention for making a medicament for treating a sex hormone dependent pathological condition.

The present invention is not intended to be limited in scope by the specific embodiments described herein. Various modifications of the invention in addition to those described herein will be apparent to those skilled in the art. Such modifications are intended to fall within the scope of the invention.

What is claimed:

1. A dihydrohalide dihydrate salt of a compound of formula I: 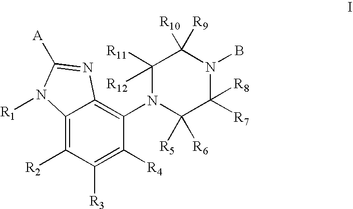
wherein:
A is aryl or heteroaryl;
B is (CR₁₃R₁₄)ₖ-D;
D is H, alkyl, cycloalkyl, heterocycloalkyl, aryl, or heteroaryl;
k is 0, 1, 2, or 3;
R₁ is H, the tautomeric form, or alkyl;
R₂, R₃, and R₄ are, independently, H, alkyl, halogen, or OR₁;
R₅, R₆, R₇, R₈, R₉, R₁₀, R₁₁, and R₁₂, are, independently, H, alkyl, alkenyl, or alkynyl;
R₁₃ and R₁₄ are, independently at each occurrence, H or alkyl.

2. The salt of claim 1, wherein the dihydrohalide dihydrate salt is crystalline.

3. The salt of claim 1, wherein the compound of formula I is 6-({4-[2-(4-tert-butylphenyl)-1H-benzimidazol-4-yl]-piperazin-1-yl}methyl)-quinoxaline.

4. The salt of claim 1, which is 6-({4-[2-(4-tert-butylphenyl)-1H-benzimidazol-4-yl]-piperazin-1-yl}methyl)-quinoxaline dihydrochloride dihydrate.

5. The salt of claim 4, wherein the dihydrochloride dihydrate salt is crystalline.

6. The salt of claim 5 exhibiting an X-ray diffraction pattern having characteristic peaks expressed in degrees 2θ at 7,981, 9,980, and 20,950.

7. The salt of claim 5 exhibiting an X-ray diffraction pattern having characteristic peaks expressed in degrees 2θ at 9,809, 9,980, and 16,399.

8. The salt of claim 5 exhibiting an X-ray diffraction pattern having characteristic peaks expressed in degrees 2θ at 9,442, 13,357, and 21,876.

9. A method comprising reacting a compound of formula I: 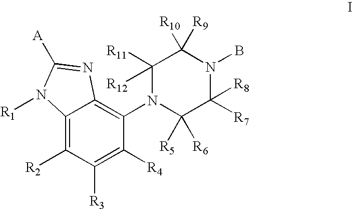
wherein:
A is aryl or heteroaryl;
B is (CR₁₃R₁₄)ₖ-D;
D is H, alkyl, cycloalkyl, heterocycloalkyl, aryl, or heteroaryl;
k is 0, 1, 2, or 3;
R₁ is H, the tautomeric form, or alkyl;
R₂, R₃, and R₄ are, independently, H, alkyl, halogen, or OR₁;
R₅, R₆, R₇, R₈, R₉, R₁₀, R₁₁, and R₁₂, are, independently, H, alkyl, alkenyl, or alkynyl;
R₁₃ and R₁₄ are, independently at each occurrence, H or alkyl;
with an alcohol, water, and an acid.

10. The method of claim 9 wherein the compound of formula I is 6-({4-[2-(4-tert-butylphenyl)-1H-benzimidazol-4-yl]-piperazin-1-yl}methyl)-quinoxaline free base.

11. The method of claim 10, wherein the alcohol is ethanol.

12. The method of claim 10, wherein the acid is hydrochloric acid.

13. A method comprising reacting a compound of formula I: 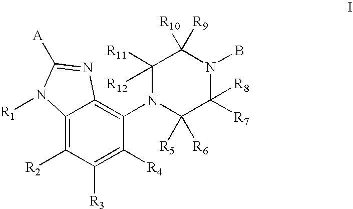
wherein:
A is aryl or heteroaryl;
B is (CR₁₃R₁₄)ₖ-D;
D is H, alkyl, cycloalkyl, heterocycloalkyl, aryl, or heteroaryl;
k is 0, 1, 2, or 3;
R₁ is H, the tautomeric form, or alkyl;
R₂, R₃, and R₄ are, independently, H, alkyl, halogen, or OR₁;
R₅, R₆, R₇, R₈, R₉, R₁₀, R₁₁, and R₁₂, are, independently, H, alkyl, alkenyl, or alkynyl;
R₁₃ and R₁₄ are, independently at each occurrence, H or alkyl;
with an alcohol, water and an acid; and seeding said reaction product with a dihydrohalide dihydrate salt of the compound of formula I.

14. The method of claim 13, wherein the compound of formula I is 6-({4-[2-(4-tert-butylphenyl)-1H-benzimidazol-4-yl]-piperazin-1-yl}methyl)-quinoxaline.

15. The method of claim 13, wherein the dihydrohalide dihydrate salt is 6-({4-[2-(4-tert-butylphenyl)-1H-benzimidazol-4-yl]-piperazin-1-yl}methyl)-quinoxaline dihydrochloride dihydrate.

16. The method of claim 13, wherein the alcohol is ethanol.

17. The method of claim 13, wherein the acid is hydrochloric acid.

18. A pharmaceutical composition comprising the compound of claim 1 and a pharmaceutically acceptable carrier or excipient.

19. A pharmaceutical composition comprising the compound of claim 2 and a pharmaceutically acceptable carrier or excipient.

20. A pharmaceutical composition comprising the compound of claim 4 and a pharmaceutically acceptable carrier or excipient.

21. A pharmaceutical composition comprising the compound of claim 5 and a pharmaceutically acceptable carrier or excipient.

22. A pharmaceutical composition comprising the compound of claim 6 and a pharmaceutically acceptable carrier or excipient.

23. A pharmaceutical composition comprising the compound of claim 7 and a pharmaceutically acceptable carrier or excipient.

24. A pharmaceutical composition comprising the compound of claim 8 and a pharmaceutically acceptable carrier or excipient.

25. A method of treating a patient suspected of suffering from a sex hormone dependent pathological condition, comprising administering to the patient an effective amount of a compound of claim 1.

26. The method of claim 25, wherein the sex hormone dependent pathological condition is prostate cancer, endometriosis, uterine fibroids, uterine cancer, breast cancer, ovarian cancer, testicular cancer, primary hirsutism, or lutenizing hormone surge.

27. A method of treating a patient suspected of suffering from a sex hormone dependent pathological condition, comprising administering to the patient an effective amount of a compound of claim 2.

28. The method of claim 27, wherein the sex hormone dependent pathological condition is prostate cancer, endometriosis, uterine fibroids, uterine cancer, breast cancer, ovarian cancer, testicular cancer, primary hirsutism, or lutenizing hormone surge.

29. A method of treating a patient suspected of suffering a sex hormone dependent pathological condition, comprising administering to the patient an effective amount of a compound of claim 3.

30. The method of claim 29, wherein the sex hormone dependent pathological condition is prostate cancer, endometriosis, uterine fibroids, uterine cancer, breast cancer, ovarian cancer, testicular cancer, primary hirsutism, or lutenizing hormone surge.

31. A method of treating a patient suspected of suffering a sex hormone dependent pathological condition, comprising administering to the patient an effective amount of a compound of claim 4.

32. The method of claim 31, wherein the sex hormone dependent pathological condition is prostate cancer, endometriosis, uterine fibroids, uterine cancer, breast cancer, ovarian cancer, testicular cancer, primary hirsutism, or lutenizing hormone surge.

33. A method of treating a patient suspected of suffering a sex hormone dependent pathological condition, comprising administering to the patient an effective amount of a compound of claim 5.

34. The method of claim 33, wherein the sex hormone dependent pathological condition is prostate cancer, endometriosis, uterine fibroids, uterine cancer, breast cancer, ovarian cancer, testicular cancer, primary hirsutism, or lutenizing hormone surge.

35. A method of treating a patient suspected of suffering a sex hormone dependent pathological condition, comprising administering to the patient an effective amount of a compound of claim 6.

36. The method of claim 35, wherein the sex hormone dependent pathological condition is prostate cancer, endometriosis, uterine fibroids, uterine cancer, breast cancer, ovarian cancer, testicular cancer, primary hirsutism, or lutenizing hormone surge.

37. A method of treating a patient suspected of suffering a sex hormone dependent pathological condition, comprising administering to the patient an effective amount of a compound of claim 7.

38. The method of claim 37, wherein the sex hormone dependent pathological condition is prostate cancer, endometriosis, uterine fibroids, uterine cancer, breast cancer, ovarian cancer, testicular cancer, primary hirsutism, or lutenizing hormone surge.
